(19) United States
(12) Patent Application Publication
(10) Pub. No.: US 2005/0154268 A1
(43) Pub. Date: Jul. 14, 2005

(54) Body component concentration measuring apparatus and method

(76) Inventors: In-duk Hwang, Suwon-si (KR); Wan-taek Han, Hwaseong-si (KR); Kye-jin Jeon, Suwon-si (KR); Kun-kook Park, Yongin-si (KR); Eun-young Choe, Suwon-si (KR); Hyun-tai Hwang, Yongin-si (KR)

Correspondence Address: LEE & STERBA, P.C., Suite 2000, 1101 Wilson Boulevard, Arlington, VA, 22209, US

(21) Appl. No.: 11/002,136

(22) Filed: Dec. 3, 2004

(30) Foreign Application Priority Data
Jan. 12, 2004 (KR) 2004-1961

(51) Int. Cl. A61B 5/00

(52) U.S. Cl. 600/316; 600/310; 600/365

(57) ABSTRACT

A body component concentration measuring apparatus, and method, for measuring a concentration of a body component in a measuring portion of a living body, includes a light source for emitting light, a diffraction grating being movable to disperse light emitted from the light source into light components having a plurality of wavelength bands, a shutter for controlling transmission of the light components having the plurality of wavelength bands, a lens for focusing the light components passing the shutter onto the measuring portion, and a detector for detecting the light components passing through the measuring portion.

### BACKGROUND OF THE INVENTION

1. Field of the Invention

The present invention relates to measuring a concentration of a body component. More specifically, the present invention relates to a body component concentration measuring apparatus and method using a scanning spectroscopic system.

2. Description of the Related Art

With overall improvements in quality of life and living conditions, interest in personal health has increased. As a result, a wide array of home medical equipment that allows people to easily monitor their personal health has been researched and developed.

In a normal human body, body fluids are organically circulated and adjusted so that quantities of the bodily fluids are maintained within constant ranges. Bodily fluids include blood, urine, interstitial fluid, sweat, saliva, and others. In particular, concentrations of body fluids, such as blood, urine (glucose and protein), are essential parameters in determining a person's state of health. In addition, concentrations of blood components, such as glucose, hemoglobin, bilirubin, cholesterol, albumin, creatinine, protein, urea, and others, play an important role in assessing a person's state of health.

When a human body is infected with a disease, a composition or a volume of a component in a bodily fluid changes, which may result in death. For example, a normal person's blood glucose concentration is about 80 mg/dl before a meal and about 120 mg/dl after a meal. In order to maintain such a normal glucose concentration, a human pancreas secretes an appropriate amount of insulin before or after the meal so that glucose can be absorbed into the liver and skeletal muscle cells. However, when the pancreas does not secrete an appropriate amount of insulin to maintain a normal blood glucose concentration due to a disease or other causes, an excessive amount of glucose exists in the blood, which causes diseases of the heart or liver, arteriosclerosis, hypertension, cataract, retinal bleeding, nerve damage, hearing loss, or visual impairment, all of which may cause serious problems including death. Accordingly, a technique of measuring a change in a bodily fluid in a human body is considered very important.

Before an extreme result, such as severe illness or death, occurs, it is critical to measure a variation in concentrations of in-vivo components. Methods of measuring the concentration of a component of bodily fluid include invasive methods of directly collecting a sample from a subject and gathering measurements on the collected part of the subject and noninvasive methods of gathering measurements without directly collecting a sample from a subject. Since invasive methods have many problems, techniques of easily analyzing components of bodily fluid using a noninvasive method have been continuously researched and developed.

Conventionally, when measuring a component of bodily fluid, for example, blood glucose, blood is extracted, reacted with a reagent, and then analyzed using a clinical analysis system or quantifying a change in the color of a test strip. When such a blood glucose test is performed daily, a patient suffers from pain resulting from the direct blood collection and is susceptible to infection. Moreover, since it is difficult to continuously monitor the blood glucose level, it is difficult to properly treat a patient in an emergency situation. In addition, use of disposable strips and reagents may be a financial burden on the patient. Furthermore, these disposable strips and reagents cause environmental contamination, and as such, require special treatment. Accordingly, development of a technique of measuring a blood glucose concentration without extracting blood is desired for monitoring and adjusting the blood glucose level of a diabetic or diagnosing a person's state of health. Many methods of noninvasively measuring blood glucose have been researched, but instruments using these methods have not been commercialized.

In most conventional, spectroscopic methods of measuring a concentration of a blood component in a human body, a part of the human body is irradiated with light within a visible and near infrared wavelength range. Light reflected from or transmitted through the human body is then detected. In such spectroscopic methods, a spectrum is usually measured to measure the concentration of a blood component. Here, a reference light source having a wavelength that responds best to a blood component to be measured and a bandwidth that effectively counterbalances an influence of an interference substance is required. Conventionally, a continuous wave (CW) lamp is used as a light source, and the intensity of the light is measured using an expensive array detector, or a spectrum is measured using a spectroscopic system, in order to calculate the concentration of a component. Alternatively, a light emitting diode (LED) or a laser diode (LD) may be used as the light source.

However, since the concentration of a component to be measured may be very low in blood and a light diffusion effect is greater than a light absorption effect in living tissue and blood, detected signals are very weak. Thus, a method of amplifying the signal is required. Moreover, since organic substances in a human body continuously flow, a component concentration can be accurately measured only when the measurement is quickly performed. In addition, it must be noted that an average energy incident on a human body should not go beyond a limit that may damage the human body. In particular, in an NIR wavelength range of 700 through 2500 nm, a glucose absorption band is widely distributed, but a maximum absorption of glucose is small as compared to a large aqueous background spectrum. Resultantly, a signal to noise ratio (SNR) is small, which makes accurate measurements very difficult.

### SUMMARY OF THE INVENTION

The present invention is therefore directed to a body component concentration measuring apparatus and method, which substantially overcome one or more of the problems due to the limitations and disadvantages of the related art.

It is a feature of an embodiment of the present invention to provide a body component concentration measuring apparatus and method that is capable of non-invasively measuring a concentration of a body component.

It is another feature of an embodiment of the present invention to provide a simple body component concentration measuring apparatus having reduced size and weight.

It is still another feature of an embodiment of the present invention to provide a body component concentration measuring apparatus and method that can improve estimation ability of glucose concentration by analyzing two light components having two wavelength bands of about 1,600 nm and 2,200 nm, which are absorbed by glucose, using a scanning spectroscopic system.

At least one of the above features and other advantages may be provided by a body component concentration measuring apparatus for measuring a concentration of a body component in a measuring portion of a living body, the apparatus including a light source for emitting light, a diffraction grating being movable to disperse light emitted from the light source into light components having a plurality of wavelength bands, a shutter for controlling transmission of the light components having the plurality of wavelength bands, a lens for focusing the light components passing the shutter onto the measuring portion, and a detector for detecting the light components passing through the measuring portion.

The body component may be glucose, the plurality of wavelength bands may be two wavelength bands. One of the two wavelength bands is about 1,600 nm and the other is about 2,200 nm.

When the shutter is opened, an amplifier gain of the light component having the wavelength band of about 2,200 nm is larger than an amplifier gain of the light component having the wavelength of about 1,600 nm.

When the shutter is opened, different gains are acquired according to the light components having the plurality of wavelength bands.

The apparatus may further include an light source unit for providing light to the diffraction grating, a high pass filter provided between the diffraction grating and the shutter, a light guide unit provided between the lens and the measuring portion of the living body, and a signal light delivery unit for delivering light from the measuring portion to the detector. The light source unit may be a tungsten halogen lamp.

The high pass filter may transmit a light component having a wavelength of about 1,200 nm or longer. The apparatus may further include a polystyrene film coupled in series to the high pass filter for calibrating wavelengths of the light components. The polystyrene film may have a thickness of about 1 mm.

The apparatus may further include a motor for rotating the diffraction grating at high speed, movement of the diffraction grating being rotary movement. The diffraction grating may have a scanning speed of about two (2) scans/second.

The apparatus may further include an amplifier for amplifying signals detected by the detector, an analog-digital converter for converting the detected signals into digital signals, a digital signal processing unit, a concentration estimating unit for estimating a concentration of the body component based on data output from the digital signal processing unit using a built-in algorithm, and a micro processor.

At least one of the above features and other advantages may be provided by a method of measuring a concentration of a body component in a measuring portion of a living body includes generating light components having a plurality of wavelength bands, acquiring different gains by the light components passing through the measuring portion by varying an amplifier gain according to the plurality of wavelength bands when a shutter is opened, and estimating the concentration of the body component using signals obtained from the different gains.

The body component may be glucose, the plurality of wavelength bands may be two wavelength bands. One of the two wavelength bands is about 1,600 nm and the other is about 2,200 nm.

When the shutter is opened, the amplifier gain of the light component having the wavelength band of about 2,200 nm is larger than that of the light component having the wavelength of about 1,600 nm.

Generating the light components may include calibrating the wavelengths of the light components using a polystyrene film coupled in series to a high pass filter. Generating the light components may include scanning a diffraction grating with a motor, the scanning being performed at high speed.

The method may further include removing the polystyrene film after calibrating the wavelengths.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1 illustrates a schematic diagram of a body component measuring apparatus including a scanning spectroscopic system according to an exemplary embodiment of the present invention; and

FIG. 2 is a graph and diagram illustrating operation of a shutter and an amplifier gain for a purpose of correcting a base line according to an exemplary embodiment of the present invention.

### DETAILED DESCRIPTION OF THE INVENTION

Korean Patent Application No. 2004-1961, filed on Jan. 12, 2004, in the Korean Intellectual Property Office, and entitled: “Body Component Measuring Apparatus and Method,” is incorporated by reference herein in its entirety.

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art.

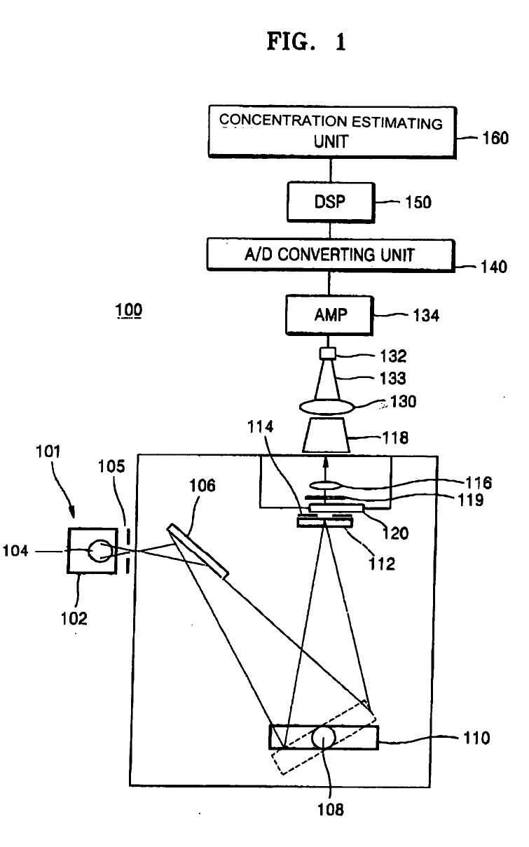

FIG. 1 illustrates a schematic diagram of a body component measuring apparatus including a scanning spectroscopic system according to an exemplary embodiment of the present invention.

A body component measuring apparatus 100 according to an exemplary embodiment of the present invention includes a light source unit 101 having a light source 104, e.g., a halogen lamp, and a power supply (not shown) in a housing 102, first and second slits 105 and 114, a reflecting mirror 106 for reflecting light emitted from the light source 104, a diffraction grating 110 for dispersing light reflected from the reflecting mirror 106, a shaft 108 for rotating the diffraction grating 110, a high pass filter 112 between the diffraction grating 110 and the second slit 114, a lens 116, e.g., a cylindrical lens, a light guide unit 118, a polystyrene film 119, a shutter 120, a detector 132, a signal light delivery unit 133 for guiding light reflected from or passing through a measuring portion 130 into the detector 132, and an amplifier 134. The measuring portion 130 may be a part of a biological tissue, e.g., a finger web or an ear lobe, where a measurement of a body component concentration is to be taken.

According to an exemplary embodiment of the present invention, the high pass filter 112 is provided between the second slit 114, which is positioned adjacent an outlet of the spectroscopic system, and the diffraction grating 110. The high pass filter 112 may preferably be fixed right below the second slit 114.

The body component measuring apparatus 100 according to an exemplary embodiment of the present invention may further include a digital signal processing (DSP) unit 150, a concentration estimating unit 160, and an analog-digital converting unit 140.

Operation of the body component concentration measuring apparatus 100 according to an exemplary embodiment of the present invention will now be described.

In operation, light emitted from the light source 104, e.g., a halogen lamp, passes through the first slit 105 provided adjacent an inlet of the spectroscopic system and then is incident on the reflecting mirror 106. Light is reflected by the reflecting mirror 106 to be incident on the diffraction grating 110, which is a light dispersing element. The diffraction grating 110 is coupled to the shaft 108, e.g., a motor shaft, of a direct current (DC) motor and is rotated by a driving unit of the motor. In an exemplary embodiment of the present invention, the diffraction grating 110 may be formed out of a concave grating.

Light dispersed by the diffraction grating 110 passes through the second slit 114 adjacent the outlet of the spectroscopic system and is focused on the light guide unit 118 through the lens 116, e.g., a cylindrical lens.

Light passing through the light guide unit 118 is incident on a sample cuvette containing a body component or the measuring portion 130, i.e., the specific portion of the living body where the measurement is to be taken, and is then measured. According to an exemplary embodiment of the present invention, because refractive indices between a surface of the biological tissue, i.e., the measuring portion, and an interface at which light contacts the surface of the biological tissue are different, it is possible to enhance the signal-to-noise ratio (SNR) by providing a medium for matching the refractive indices therebetween.

The signal light reflected or transmitted from the measuring portion 130 is sent to the detector 132 through the signal light delivery unit 133 having a lens, a mirror, or combination thereof.

The analog signal detected by the detector 132 is amplified by the amplifier 134.

The body component measuring apparatus 100 according to an exemplary embodiment of the present invention may further include an analog-digital converter, a digital signal processing unit, a concentration estimating unit, and a microprocessor.

The amplified analog signal is converted into a digital signal, which can be processed by the microprocessor, e.g., a microcomputer, through the analog-digital converter. The signal output from the analog-digital converter is then processed and analyzed by the digital signal processing unit. A concentration of the body component is estimated by the concentration estimating unit, which has a built-in algorithm for estimating concentrations of body components to be measured, based on data output from the digital signal processing unit. The microprocessor controls all of the processes of the system, such as emitting and receiving light, estimating the concentration, and others.

The concentration estimating unit prepares an estimation model equation capable of accurately estimating a concentration of the body component by comparing and analyzing degrees of absorption by the wavelengths used for measurement, thereby calculating the concentration of the body component being measured. Various known analyzing methods can be used for the statistical analysis for estimating the concentration.

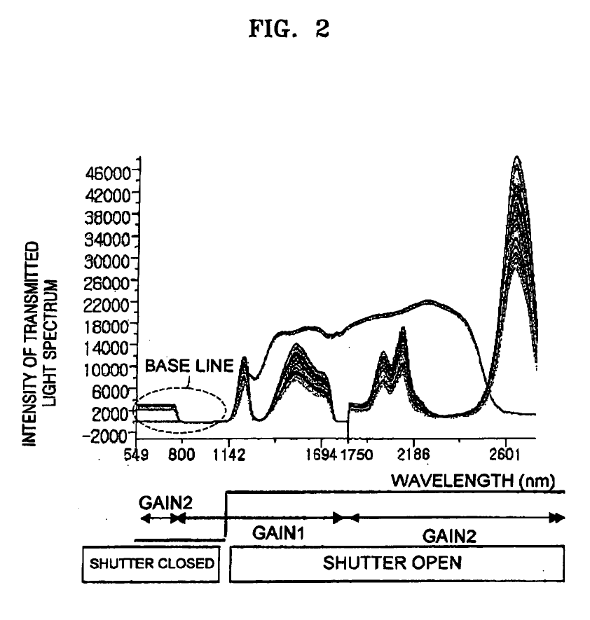

FIG. 2 is a graph and diagram illustrating operation of the shutter and an amplifier gain for a purpose of correcting a base line according to an exemplary embodiment of the present invention. A portion corresponding to the base line is shown in FIG. 2.

For the purpose of calibrating wavelengths, a polystyrene film is provided between the cylindrical lens 116 and the second slit 114 under control of the DC motor, and a measured spectrum is calculated by the built-in software, thereby calculating accurate wavelengths.

The polystyrene film according to an exemplary embodiment of the present invention has an absorption peak at a specific wavelength, e.g., 1680.90 nm, 2166.72 nm, 2306.10 nm, etc. The polystyrene film may preferably be formed to a thickness of about 1 mm.

In an exemplary embodiment of the present invention, glucose, which is one of body components, is exemplarily measured. To acquire a more intensive signal at 2,200 nm, which is an absorption band of glucose, an amplifier gain is controlled to be different from that of a wavelength band of 1,600 nm. Such an operation will now be described.

Actual absorbance is calculated by first measuring the transmitted light with a reference gain in the absence of a measuring portion and then measuring the transmitted light in the presence of the measuring portion. During the measurement, the shutter 120 is positioned between and removed from between the cylindrical lens 116 and the second slit 114 at a predetermined time interval under control of the DC motor.

As shown in FIG. 2, the shutter 120 blocking light is closed initially, and is opened when a wavelength of about 1,200 nm is incident thereon when the wavelength is scanned from a small wavelength side to a large wavelength side with rotation of the diffraction grating 110. Since intensity of light that the measuring portion transmits is still smaller at a wavelength band of about 2,200 nm than that at a wavelength band of about 1,600 nm, amplifier gain should be controlled to be still larger at the wavelength band of about 2,200 nm than at the wavelength band of about 1,600 nm, thereby more accurately estimating the concentration of blood glucose.

Therefore, because the amplifier gain changes from Gain1to Gain2 during a single scan, that is, one rotation of the diffraction grating 110, a variation of a base line occurs, as shown in the graph in FIG. 2. Such a variation of the base line due to change of the amplifier gain requires correction. Signals corresponding to the first gain Gain1 at the wavelength band of 1,600 nm and the second gain Gain2 at the wavelength band of 2,200 nm are measured in synchronism with the opening and closing of the shutter 120, the base line is corrected, and then a final absorbance is calculated. To estimate the concentration of body components, various absorbance data measured according to a concentration change of a specific body component are used. In particular, the concentration of a target body component is estimated using a conventional statistical analysis method.

The body component measuring apparatus 100 according to an exemplary embodiment of the present invention can be effectively used in various similar application systems as well as in measuring body component concentration.

According to an exemplary embodiment of the present invention, light having a wavelength of between about 1,200 nm to 2,400 nm may be used for measuring a concentration of blood glucose.

Further, a tungsten halogen lamp, which generates continuous spectra at an area ranging from visible to near-infrared light, may be used as the light source. A high pass filter transmitting only light components having a wavelength band of about 1,200 nm or longer may be provided right below the second slit 114 in the spectroscopic system to reduce a temperature rise at the measuring portion and to avoid use of an order sorting filter.

Furthermore, according to an exemplary embodiment of the present invention, the detector 132 performs the correction of the base line value in accordance with one or more different values of amplifier gain by controlling the shutter 120 to block light components having a wavelength of 1,200 nm or shorter.

Furthermore, according to an exemplary embodiment of the present invention, calibration of wavelength is performed using a structure in which the high pass filter and the polystyrene film are coupled in series. After completing the calibration of wavelength, the polystyrene film is removed and only the high pass filter 122 is positioned in the optical path.

Furthermore, the scanning speed of the diffraction grating 110 provided in the spectroscopic system is preferably set to about 2 scan/sec. This is because it is necessary that the measuring time should be reduced as much as possible to minimize influence of continuous circulation of in-vivo organic substances.

Furthermore, according to an exemplary embodiment of the present invention, by allowing light components incident on the measuring portion to have specific wavelength bands through scanning the diffraction grating, it is possible to non-invasively measure the concentration of the body component.

In an exemplary embodiment of the present invention, in order to measure the concentration of glucose, a wide-band spectrum including a wavelength band to be largely absorbed by glucose and a wavelength band to be minimally absorbed by glucose is obtained by rotating the diffraction grating, thereby dispersing the light incident thereon. First, the dispersed light components are applied to the measuring portion. Then, by statistically analyzing the absorption differences of signal lights reflected or transmitted from the measuring portion, it is possible to estimate the concentration of blood glucose accurately.

Furthermore, in an exemplary embodiment of the present invention, since an intensity of a light component having a wavelength of about 2,200 nm, which is an absorption wavelength band of glucose, is several tens times smaller than the intensity of a light component having a wavelength of about 1,600 nm after light components passes through the measuring portion, the amplifier gain has different values between the two wavelength bands, thereby facilitating enhancement of the signal-to-noise ratio and the resolution.

As described above, according to an embodiment of the present invention, by applying different amplifier gain values to two specific wavelength bands, it is possible to improve accuracy of estimating a body component concentration.

Further, according to the exemplary embodiment of the present invention, by scanning the diffraction grating at high speed, it is possible to reduce measuring time. In addition, since only light components required for estimating the concentration of a body component are incident on the measuring portion, it is possible to minimize the variation of measurement due to the temperature rise of the measuring portion.

Furthermore, according to the exemplary embodiment of the present invention, it is possible to non-invasively measure the concentration of a body component. Further, it is possible to provide a simple body component concentration measuring apparatus having reduced size and weight.

Exemplary embodiments of the present invention have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

1. A body component concentration measuring apparatus for measuring a concentration of a body component in a measuring portion of a living body, the apparatus comprising:
a light source for emitting light;
a diffraction grating being movable to disperse light emitted from the light source into light components having a plurality of wavelength bands;
a shutter for controlling transmission of the light components having the plurality of wavelength bands;
a lens for focusing the light components passing the shutter onto the measuring portion; and
a detector for detecting the light components passing through the measuring portion.

2. The apparatus according to claim 1, wherein when the body component is glucose, the plurality of wavelength bands is two wavelength bands.

3. The apparatus according to claim 2, wherein one of the two wavelength bands is about 1,600 nm and the other is about 2,200 nm.

4. The apparatus according to claim 3, wherein when the shutter is opened, an amplifier gain of the light component having the wavelength band of about 2,200 nm is larger than an amplifier gain of the light component having the wavelength of about 1,600 nm.

5. The apparatus according to claim 1, wherein when the shutter is opened, different gains are acquired according to the light components having the plurality of wavelength bands.

6. The apparatus according to claim 1, further comprising:
an light source unit for providing light to the diffraction grating;
a high pass filter provided between the diffraction grating and the shutter;
a light guide unit provided between the lens and the measuring portion of the living body; and
a signal light delivery unit for delivering light from the measuring portion to the detector.

7. The apparatus according to claim 6, wherein the light source unit is a tungsten halogen lamp.

8. The apparatus according to claim 6, wherein the high pass filter transmits a light component having a wavelength of about 1,200 nm or longer.

9. The apparatus according to claim 6, further comprising a polystyrene film coupled in series to the high pass filter for calibrating wavelengths of the light components.

10. The apparatus according to claim 9, wherein the polystyrene film has a thickness of about 1 mm.

11. The apparatus according to claim 1, further comprising a motor for rotating the diffraction grating at high speed, movement of the diffraction grating being rotary movement.

12. The apparatus according to claim 11, wherein the diffraction grating has a scanning speed of about two (2) scans/second.

13. The apparatus according to claim 1, further comprising:
an amplifier for amplifying signals detected by the detector;
an analog-digital converter for converting the detected signals into digital signals;
a digital signal processing unit;
a concentration estimating unit for estimating a concentration of the body component based on data output from the digital signal processing unit using a built-in algorithm; and
a micro processor.

14. A method of measuring a concentration of a body component in a measuring portion of a living body, comprising:
generating light components having a plurality of wavelength bands;
acquiring different gains by the light components passing through the measuring portion by varying an amplifier gain according to the plurality of wavelength bands when a shutter is opened; and
estimating the concentration of the body component using signals obtained from the different gains.

15. The method according to claim 14, wherein when the body component is glucose, the plurality of wavelength bands is two wavelength bands.

16. The method according to claim 15, wherein one of the two wavelength bands is about 1,600 nm and the other is about 2,200 nm.

17. The method according to claim 16, wherein when the shutter is opened, the amplifier gain of the light component having the wavelength band of about 2,200 nm is larger than that of the light component having the wavelength of about 1,600 nm.

18. The method according to claim 14, wherein generating the light components comprises calibrating the wavelengths of the light components using a polystyrene film coupled in series to a high pass filter.

19. The method according to claim 18, further comprising removing the polystyrene film after calibrating the wavelengths.

20. The method according to claim 14, wherein generating the light components comprises scanning a diffraction grating with a motor, the scanning being performed at high speed.
